(19) United States
(12) Patent Application Publication
(10) Pub. No.: US 2017/0150745 A1
(43) Pub. Date: Jun. 1, 2017

(54) METHODS OF EXTRACTION AND PURIFICATION OF LUO HAN GUO MOGROSIDE V, NATURAL SWEETENER COMPOSITIONS THEREWITH AND USES OF SAID COMPOSITION

(71) Applicant: GLG Life Tech Corporation, Richmond (CA)

(72) Inventors: Yong Luke Zhang, Richmond (CA); Cunbiao Kevin Li, Richmond (CA)

(21) Appl. No.: 15/309,629

(22) PCT Filed: May 8, 2015
(86) PCT No.: PCT/CA2015/000308

§ 371 Date: Nov. 8, 2016

(30) Foreign Application Priority Data
May 8, 2014 (CN) 201410190403.7

(51) Int. Cl. A23L 27/12; C07J 17/00; B01D 15/18; B01D 15/26; A23L 2/60; A23L 33/105

(52) U.S. Cl. CPC A23L 27/12; A23L 2/60; A23L 33/105; B01D 15/1871; B01D 15/265; C07J 17/005; A23V 2002/00

(57) ABSTRACT

A method for purifying Mogroside V comprises passing a solution of a pre-prepared Siraitia grosvenori primary extract through a multi-column system including a plurality of columns, in series, packed with a porous adsorbent resin to provide at least one column having adsorbed mogrosides and eluting fractions with Mogroside V content from the at least one column having absorbed mogrosides to provide an eluted solution with Mogroside V content.

### FIELD OF THE INVENTION

The present invention relates generally to methods of extracting natural products from plants, in particular from fresh Monk fruit.

### BACKGROUND

In the food and beverage industry, there is a general preference for the consumption of sweet foods, and manufacturers and consumers commonly add sugar in the form of sucrose (table sugar), fructose or glucose to beverages, food, etc. to increase the sweet quality of the beverage or food item. Although most consumers enjoy the taste of sugar, sucrose, fructose and glucose are high calorie sweeteners. Many alternatives to these high calorie sweeteners are artificial sweeteners or sugar substitutes, which can be added as an ingredient in various food items.

Common artificial sweeteners include saccharin, aspartame, and sucralose. Unfortunately, these artificial sweeteners have been associated with negative side effects. Therefore, alternative, natural non-caloric or low-caloric or reduced caloric sweeteners have been receiving increasing demand as alternatives to the artificial sweeteners and the high calorie sweeteners comprising sucrose, fructose and glucose. Like some of the artificial sweeteners, these alternatives provide a greater sweetening effect than comparable amounts of caloric sweeteners; thus, smaller amounts of these alternatives are required to achieve sweetness comparable to that of sugar. These alternative, natural sweeteners, however, can be expensive to produce and/or possess taste characteristics different than sugar (such as sucrose), including, in some instances, undesirable taste characteristics such as sweetness linger, delayed sweetness onset, negative mouth feels and different taste profiles, such as off-tastes, including bitter, metallic, cooling, astringent, licorice-like tastes.

Luo Han Guo (luohanguo) refers to the fruit of Siraitia grosvenori, formerly called Momordica grosvenori, a member of the Curcubitaceae¹. The fruit is well-known for its sweet taste; this plant family (Gourd family) has other members that contain remarkable sweet components, including additional species of the genus Siraitia (e.g., S. siamensis, S. silomaradjae, S. sikkimensis, S. africana, S. borneensis, and S. taiwaniana²) and the popular herb jiaogulan (Gynostemma pentaphyllum). The latter herb, which has both sweet and bitter tasting triterpene glycosides in its leaves, is now sold worldwide as a tea and made into an extract for use in numerous health-care products. Luohanguo has been used as a medicinal herb for treating cough and sore throat and is popularly considered, in southern China, to be a longevity aid. These are the same uses as listed for jiaogulan. Luohanguo has more recently been developed into a non-caloric sweetener to compete with other herbal sweeteners such stevioside from the unrelated Stevia leaf. ¹ Ling Yeouruenn, A New Compendium of Materia Medica, 1995 Science Press, Beijing² Dawson G E, et al., Process and composition for sweet juice from Cucurbitaceae fruit, U.S. Pat. No. 5,411,755, May 2, 1995

Luohanguo is collected as a round green fruit that turns brown upon drying. The sweet taste of luohanguo comes primarily from mogrosides, a group of terpene glycosides, present at the level of about 1% of the fleshy part of the fruit³. Both the fresh and dried fruits are extracted to yield a powder that is about 80% mogrosides. The mogrosides have been numbered, 1-5, and the main component is called mogroside-5, previously known as esgoside. Other, similar compounds from luohanguo have been labeled siamenoside and neomogroside. The mixed mogrosides are estimated to be about 300 times as sweet as sugar by weight, so that the 80% extracts are nearly 250 times sweeter than sugar; pure mogrosides 4 and 5 may be 400 times as sweet as sugar by weight. ³ Hsu H Y, et al., Oriental Materia Medica, 1986 Oriental Healing Arts Institute, Long Beach, Calif.

The mogrosides are highly stable molecules based on a cucurbitane skeleton. As the name suggests, the cucurbitacins occur predominately in the family Cucurbitaceae, and can be found in many groups of plants in the cucumber family, of which Luo Han is a member. Cucurbitacins are a group of secondary plant metabolites, classified chemically as triterpenes based on the 19-(10→9β)-abeo-10α-lanost-5-ene (cucurbitane) skeleton. All terpenoids are derived from repetitive fusion of branched 5-carbon isoprene units; the triterpenoids, which contain 30 carbon atoms, are generated by the head-to-head joining of the two C15 chains, each of which contains 3 isoprene units joined head to tail.

Mogrosides are formed of varying numbers of glucose units, from 2 to 6, attached to carbon 3 and carbon 24 (indicated as R1 and R2 in FIG. 1) on the triterpene backbone.

All of the mogrosides are classified as triterpene glucosides, designated as the diglucoside, triglucoside, tetraglucoside, pentaglucoside, and hexaglucoside. Mogrosides IV, V, and VI are very sweet and are responsible for the sweetness of Luo Han fruit and consequently that of the PureLo® concentrate of Luo Han fruit. Mogroside V is the major sweetness component of the fruit, comprising up to 0.5% of the dried fruit weight. The inherent robust stability of the coordinate covalent bonds between the triterpene framework and carbohydrate residues attached at carbons 3 and 24 render the mogrosides inert to thermal and enzymatic degradation. Thus mogrosides are biochemically stable, non-nutritive, and non-hygroscopic¹.

The simplest mogroside, mogroside II, has one glucose residue attached to each of carbons 3 and 24. Mogroside III differs in having an additional glucose residue chained to carbon 24, while mogroside IV has 2-unit glucose side chains at both carbon 3 and 24. This progression continues through mogroside VI, which has 3 glucose residues attached to each of the two carbons at locations 3 and 24 of the triterpene backbone. This is illustrated below in FIG. 2.

The molecular formula of mogroside V is C060H102029; its molecular weight is 1286 Dalton (Da). The molecular formulas of the other mogrosides are as follows:

mogroside II: C₄₂H₇₂O₁₄

mogroside III: C₄₈H₈₂O₁₉

mogroside IV: C₅₄H₉₂O₂₄

mogroside VI: C₆₆H₁₁₂O₃₄

The structural formula of mogroside V is shown in FIG. 3.

The fruit itself, though sweet, has too many additional flavors that would make it unsuitable for widespread use as a sweetener, so the key is processing it to eliminate the undesired flavors. The fruit is seldom used fresh due to the problems of storing it and the raw fruit has unattractive flavors and a tendency to easily form off-flavors by fermentation. Also, its pectin eventually gels. So, it is common practise in China to dry the fruits for any further use, and in fact this is how they appear in Chinese herb shops. The fruits are slowly dried in ovens; the drying process preserves the fruit and removes some of the objectionable flavor of the fresh fruit, which is associated with volatile components. Unfortunately, the drying also causes the formation of bitter, astringent flavors. These flavors limit the use of the dried fruits and dried fruit extracts to the preparation of dilute teas and soups and products to which sugar, honey, and the like are added. It is an ongoing problem with the use of Mogroside V as a sweetener.

In addition, although there are many methods of extracting Mogroside V, unstable mass of Mogrosides still exist which directly affects the use by consumers; for example, differences in taste, content, and color affect the market promotion of Mogrosides.

While there is increasing commercial interest in Mogroside V and its natural sweetening properties, there are a number of limiting factors in their use, including, for some, bitter taste, varying sweetening capabilities and extraction costs/difficulties. It is an object of the present invention to obviate or mitigate the above and other disadvantages.

### SUMMARY OF THE INVENTION

It is an object of the present invention to provide a method for extracting the Mogroside V from the plant Siraitia grosvenori with high purity, excellent color and pure taste.

The present invention further provides a natural extract comprising Mogroside V, which is extracted and purified from the plant material according the methods described and claimed herein

The present invention further provides a natural sweetener composition comprising extracts of Mogroside V, which are extracted and purified from the plant material as described herein.

The present invention further provides foods, beverages, nutraceuticals, functional foods, medicinal formulations, cosmetics, health products, condiments and seasonings comprising extracts of Mogroside V extracted and purified from any of the plant material as described herein.

The present invention further provides a natural sweetener composition comprising a blend of Mogroside V extracted and purified from any of the plant material as described herein, along with at least one steviol glycoside.

The natural sweetener compositions of the present invention may be zero calories or merely reduced calorie, as desired. As such, full-calorie, mid-calorie, low-calorie and zero-calorie beverages containing Mogroside V extract or the sweetener compositions of the present invention are also provided.

What the present invention provides are compositions of Mogroside V which achieve benefits and advantages above and beyond the prior extracts of Mogroside V. These natural sweetener compositions have a taste profile comparable to sugar, are desired, are not prohibitively expensive to produce and can be added, for example, to beverages and food products to satisfy consumers looking for a sweet taste. As such, these compositions allow for the customization of sweetening goals. In particular, the method of extraction, provided herein, enables the cost effective and hence commercially viable production of plant extracts comprising Mogroside V.

The present invention provides a method for purifying Mogroside V including passing a solution of a pre-prepared Siraitia grosvenori primary extract through a multi-column system including a plurality of columns, in series, packed with a porous adsorbent resin to provide at least one column having adsorbed mogrosides and eluting fractions with Mogroside V content from the at least one column having absorbed mogrosides to provide an eluted solution with Mogroside V content.

As the solution of the pre-prepared Siraitia grosvenori primary extract, comprising a plurality of mogrosides, passes through the multi-column system, the various mogrosides separate into different portions of different columns. The portions differ from each other both by total mogroside content and individual mogroside content. Fractions containing Mogroside V content are eluted/desorbed from the multi-column system separately from fractions containing low or no Mogroside V content.

Optionally, the method includes one or more additional steps. In one embodiment, the method includes washing the multi-column system with a washing solution prior to eluting fractions with high Mogroside V content in order to remove impurities.

In another embodiment, the method optionally includes decolorizing the eluted solution with high Mogroside V content, removing the alcohol solvent and passing the remaining solution through a column with macroporous adsorbent to provide a second adsorption solution.

In another embodiment, the method optionally includes deionizing the eluted solution of mogrosides. Removal of the remaining solvent from the eluted solution—optionally decolorized and/or deionized—provides a highly purified mogroside mixture.

The method of the present invention also includes preparing a Siraitia grosvenori primary extract, suitable for passage through the porous resin columns. In one embodiment, the primary extract is prepared by providing fruit of the Siraitia grosvenori plant, washing and mashing the fruit (while removing seeds), saccharifying the mashed fruit to hydrolyze the polysaccharides, extracting the saccharified matter, filtering and concentrating the extract, and then centrifuging the concentrated extraction filtrate. The primary extract is ready for passage, as a feed liquor, through the plurality of macroporous resin adsorption columns in series. The resultant product of the treatment through the resin adsorption columns in series, and resolution of those columns is referred to herein as an intermediate Mogroside V extract.

The method of the present invention also includes further processing of the intermediate Mogroside V extract. The intermediate Mogroside V extract maybe purified to remove colour, salt and impurities. This may be achieved by membrane filtration, ion exchange chromatography or activated carbon treatment (or any combinations of those).

According to one aspect of the invention, a method for producing purified Mogroside V comprises the steps of: preparing a Siraitia grosvenori primary extract, suitable for passage through the porous resin columns by washing and mashing the fruit (while removing seeds), saccharifying the mashed fruit to hydrolyze the polysaccharides, extracting the saccharified matter, filtering and concentrating the extract, centrifuging the concentrated extraction filtrate passing the concentrated extraction filtrate feed over a series of columns packed with macroporous resin and eluting mogrosides to provide eluates containing high Mogroside V and decolorizing the solutions; evaporating and deionizing; concentrating by nano-filters and drying.

These and other objects and advantages of the present invention will become more apparent to those skilled in the art upon reviewing the description of the preferred embodiments of the invention, in conjunction with the figures and examples. A person skilled in the art will realize that other embodiments of the invention are possible and that the details of the invention can be modified in a number of respects, all without departing from the inventive concept. Thus, the following drawings, descriptions and examples are to be regarded as illustrative in nature and not limiting.

### BRIEF DESCRIPTION OF THE DRAWINGS

Embodiments of the present invention will now be described, by way of example only, with reference to the attached Figures, wherein:

FIG. 1 is a molecular structure of the triterpene backbone.

FIG. 2 is the molecular structure of Mogroside II through IV side chains.

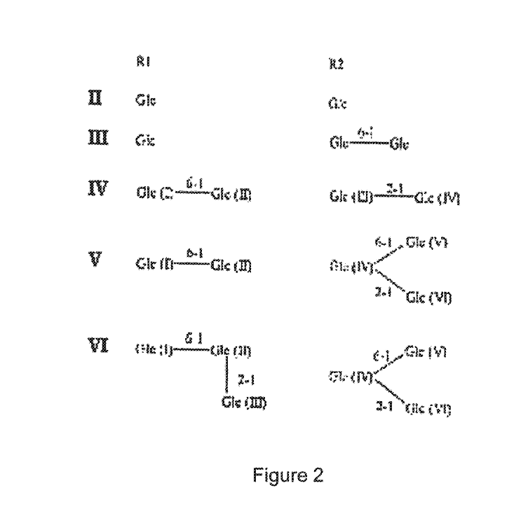

FIG. 2a is the molecular structure of Mogroside V.

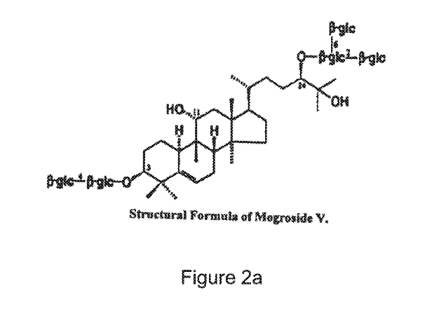

FIG. 3 is a flow diagram of the extraction process for extracting and purifying Mogroside (and preferably Mogroside V) from the Luo Han Guo fruits.

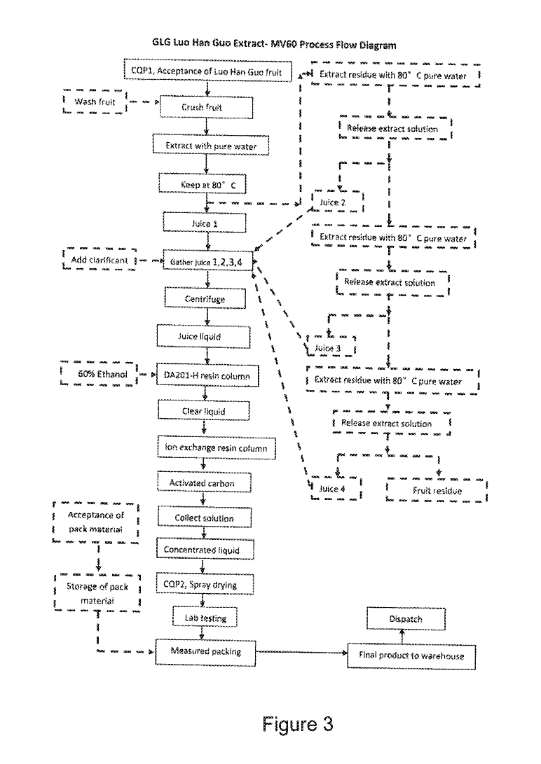

FIG. 4 shows the HPLC trace of mogrosides including Mogroside V (30 wt % in extract).

FIG. 4a shows the HPLC trace of mogrosides including Mogroside V (30 wt % in extract).

FIG. 5 shows the HPLC trace of mogrosides including Mogroside V (60 wt % in extract).

FIG. 5a shows the HPLC trace of mogrosides including Mogroside V (60 wt % in extract).

FIG. 6 shows HPLC trace of Mogroside V standard.

FIG. 7 is a graph showing indes of swett feeling of the sweetening compositions.

FIG. 8 shows the color depth and odor of powder.

FIG. 9 shows the color shade, sweetness feeling and size of product solution.

FIG. 10 shows the sweetness curve chart and time duration sensory profiles of RA97, MV50, Sucrose and dream sweetener with MV50 and RA97.

FIG. 11 shows the sweetness curve chart and time duration sensory profiles of RA97, MV25, MV50, MV55 and Sucrose.

FIG. 12 shows the sweetness curve chart and time duration sensory profiles of six samples.

FIG. 13 shows the line sensory profiles of RA97, Dream sweetener (RA97+MV50) and Sucrose.

FIG. 14 shows line sensory profiles of RA97, MV25, MV50, MV55 and Sucrose.

FIG. 15 shows line sensory profiles of RA97, Sucrose, Dream sweetener, MV25 and MV50.

FIG. 16 shows the spider plot of Dream sweetener, RA97, Sucrose, MV25 and MV50.

FIG. 17 shows the off flavor attributes of Dream sweetener, RA97, Sucrose, MV25 and MV50.

FIG. 18 shows the mouthfeeling index of all products the Sucrose, MV25, MV50, MV55, RA97 and Dream sweetener (RA97+MV50).

### DETAILED DESCRIPTION OF THE INVENTION

A detailed description of one or more embodiments of the invention is provided below along with accompanying figures that illustrate the principles of the invention. As such this detailed description illustrates the invention by way of example and not by way of limitation. The description will clearly enable one skilled in the art to make and use the invention, and describes several embodiments, adaptations, variations and alternatives and uses of the invention, including what we presently believe is the best mode for carrying out the invention. It is to be clearly understood that routine variations and adaptations can be made to the invention as described, and such variations and adaptations squarely fall within the spirit and scope of the invention.

In other words, the invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Certain definitions used in the specification are provided below. Also in the examples which follow, a number of terms are used. In order to provide a clear and consistent understanding of the specification and claims, the following definitions are provided:

The terms “an aspect”, “an embodiment”, “embodiment”, “embodiments”, “the embodiment”, “the embodiments”, “one or more embodiments”, “some embodiments”, “certain embodiments”, “one embodiment”, “another embodiment” and the like mean “one or more (but not all) embodiments of the disclosed invention(s)”, unless expressly specified otherwise.

The term “variation” of an invention means an embodiment of the invention, unless expressly specified otherwise. A reference to “another embodiment” or “another aspect” in describing an embodiment does not imply that the referenced embodiment is mutually exclusive with another embodiment (e.g., an embodiment described before the referenced embodiment), unless expressly specified otherwise.

In this specification the terms “comprise, comprises, comprised and comprising” and the terms “include, includes, included and including” are deemed to be totally interchangeable and should be afforded the widest possible interpretation.

The articles “a” and “an” are used herein to refer to one or to more than one (i.e., to at least one) of the grammatical object of the article. By way of example, “an element” means one element or more than one element.

The term “or” as used herein should be understood to mean “and/or”, unless the context clearly indicates otherwise.

The term “plurality” means “two or more”, unless expressly specified otherwise.

The term “herein” means “in the present application, including anything which may be incorporated by reference”, unless expressly specified otherwise.

The term “whereby” is used herein only to precede a clause or other set of words that express only the intended result, objective or consequence of something that is previously and explicitly recited. Thus, when the term “whereby” is used in a claim, the clause or other words that the term “whereby” modifies do not establish specific further limitations of the claim or otherwise restricts the meaning or scope of the claim.

The term “e.g.” and like terms mean “for example”, and thus does not limit the term or phrase it explains. For example, in a sentence “the image of an item is captured by an image capture device, for example a camera, the term “for example” explains that “camera” is an example of “an image capture device” through which one aspect of the data collection of this invention operates.

The term “respective” and like terms mean “taken individually”. Thus if two or more things have “respective” characteristics, then each such thing has its own characteristic, and these characteristics can be different from each other but need not be.

The term “i.e.” and like terms mean “that is”, and thus limits the term or phrase it explains.

As used herein, unless specifically indicated otherwise, the word “or” is used in the “inclusive” sense of “and/or” and not the “exclusive” sense of “either/or

The term process may be used interchangeably with method, as referring to the steps of processing as described and claimed herein.

The term Mogroside V may be used interchangeably with esgoside and has the chemical structure as noted above.

As used herein, the term “about” in connection with a measured quantity, refers to the normal variations in that measured quantity, as expected by a skilled artisan making the measurement and exercising a level of care commensurate with the objective of measurement.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values.

### Extraction and Purification

The present invention provides a method for extracting the Mogroside V with high purity, excellent color and pure taste.

Typically, Mogroside V is the most abundant single Mogroside component of Luo Han Guo extracts, accompanied by other Mogrosides such as Mogrosides I, II, III, IV and VI as well as other extracted materials, such as polyphenols, flavonoids, melanoidins, terpenes, proteins, sugars, aromatic glycosides, and semi-volatile organic compounds. In some embodiments of the invention, the Mogroside V is provided in the form of a Luo Han Guo extract (purified and concentrated to increase Mogroside V content).

In another aspect of the invention, a method for purifying Mogroside V comprises:
(a) passing a primary solution comprising mogrosides through a multi-column system including a plurality of columns packed with a porous adsorbent resin to provide at least one column having adsorbed mogrosides; and
(b) eluting fractions with Mogroside V content from the at least one column having adsorbed mogrosides to provide an eluted solution with Mogroside V content.

In another aspect of the invention, a method for purifying Mogroside V comprises:
(a) passing a primary solution comprising mogrosides through a multi-column system including a plurality of columns in series packed with an adsorbent resin to provide at least one column having adsorbed mogrosides;
(b) removing impurities from the multi-column system; and
(c) eluting fractions with Mogroside V content from the at least one column having adsorbed mogrosides to provide an eluted solution with Mogroside V content.

In another embodiment, a method for purifying Mogroside V comprises:
(a) passing a primary solution comprising mogrosides through a multi-column system including a plurality of columns in series packed with an adsorbent resin to provide at least one column having adsorbed mogrosides;
(b) eluting fractions with Mogroside V content from the at least one column having adsorbed mogrosides to provide an eluted solution with Mogroside V content;
(c) decolorizing the eluted solution with Mogroside V content to provide a first adsorption solution; and
(d) removing the alcoholic solvent from the first adsorption solution and passing the remaining solution through a column with a macroporous adsorbent to provide a second adsorption solution.

In another embodiment, a method for purifying Mogroside V comprises:
(a) passing a primary extract solution comprising of mogrosides through a multi-column system including a plurality of columns in series packed with an adsorbent resin to provide at least one column having adsorbed mogrosides;
(b) removing impurities from the multi-column system;
(c) eluting fractions with Mogroside V content from the at least one column having adsorbed mogrosides to provide an eluted solution with Mogroside V content;
(d) decolorizing the eluted solution with Mogroside V content to provide a first adsorption solution; and
(e) removing the alcoholic solvent from the first adsorption solution and passing the remaining solution through a column with a macroporous adsorbent to provide a second adsorption solution.

In another embodiment, a method for purifying Mogroside V comprises:
(a) passing a primary solution comprising mogrosides through a multi-column system including a plurality of columns in series packed with an adsorbent resin, to provide at least one column having adsorbed mogrosides;
(b) eluting fractions with Mogroside V content from the at least one column having adsorbed mogrosides to provide an eluted solution with Mogroside V content; and
(c) deionizing the solution.

In one aspect, the method of the invention comprises preparing a Siraitia grosvenori primary extract, suitable for passage through the porous resin columns. Preferably, the primary extract is prepared by providing fruit of the Siraitia grosvenori plant, washing and mashing the fruit (while removing seeds), saccharifying the mashed fruit to hydrolyze the polysaccharides, extracting the saccharified matter, filtering and concentrating the extract, and then centrifuging the concentrated extraction filtrate. The primary extract is ready for passage, as a feed liquor, through the plurality of macroporous resin adsorption columns in series.

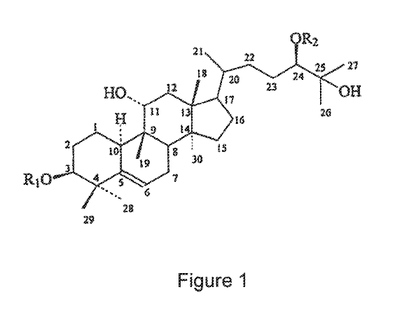

More specifically, and with reference and more specificity to FIG. 1, the method comprises:
1) washing fresh Siraitia grosvenori fruits;
2) crushing washed fruits to form crushed fruits solution.
3) saccharifying the crushed matter to hydrolyze polysaccharides to soluble sugars, forming saccharified matter;
4) extracting the saccharified matter with water, filtering extract to form an extract filtrate and then concentrating the extract filtrate to form a concentrated extraction filtrate; and
5) centrifuging the concentrated extraction filtrate.

In another aspect, the method of the invention comprises a method for purifying Mogroside V including passing a solution of a pre-prepared Siraitia grosvenori primary extract through a multi-column system including a plurality of columns, in series, packed with a porous adsorbent resin to provide at least one column having adsorbed mogrosides and eluting fractions with high Mogroside V content from the at least one column having absorbed mogrosides to provide an eluted solution with Mogroside V content (the “intermediate Mogroside V extract”).

As the solution of the pre-prepared Siraitia grosvenori primary extract, comprising a plurality of mogrosides, passes through the multi-column system, the various mogrosides separate into different portions of different columns. The portions differ from each other both by total mogroside content and individual mogroside content. Fractions containing high Mogroside V content are eluted/desorbed from the multi-column system separately from fractions containing low Mogroside content.

More specifically, the method comprises separating the intermediate Mogroside V extract by passage of the concentrated extraction filtrate through a plurality of resin adsorption columns, in series connection, to form a separated filtrate that is the desired intermediate Mogroside V extract.

The method of the present invention also includes further optional processing of the intermediate Mogroside V extract. The intermediate Mogroside V extract maybe purified to remove colour, salt and impurities. This may be achieved by membrane filtration, ion exchange chromatography or activated carbon treatment (or any combinations of those).

Preferably, the intermediate Mogroside V extract (a separated filtrate) is then purified and then concentrated to form a concentrated separated filtrate. By way of example, the concentrated separated filtrate maybe further purified in a gel column to be remove impurities and homologs of Mogroside. The resulting product from the gel column may be concentrated and/or spray dried to a final purified product.

Wherein compounds are required to be separated in accordance with the method of the invention, separation can be achieved by any suitable means including, but not limited to, gravity filtration, a plate-and-frame filter press, cross flow filters, screen filters, Nutsche filters, belt filters, ceramic filters, membrane filters, microfilters, nanofilters, ultrafilters or centrifugation. Optionally various filtration aids such as diatomaceous earth, bentonite, zeolite etc., may be used in this process.

Wherein solutions are to be treated by ion exchange resins in accordance with the method of the invention, such deionization by any suitable method including, for example, electrodialysis, filtration (nano- or ultra-filtration), reverse osmosis, ion exchange, mixed bed ion exchange or a combination of such methods.

The cation-exchange resin can be any strong acid cation-exchanger where the functional group is, for example, sulfonic acid. Suitable strong acid cation-exchange resins are known in the art and include, but are not limited to, Rohm & Haas Amberlite® 10 FPC22H resin, which is a sulfonated divinyl benzene styrene copolymer, Dowex® ion exchange resins available from Dow Chemical Company, 15 Serdolit® ion exchange resins available from Serva Electrophoresis GmbH, T42 strong acidic cation exchange resin and A23 strong base an ion exchange resin available from Qualichem, Inc., and Lewatit strong ion exchange resins available from Lanxess. In a particular embodiment, the strong acid cation-exchange resin is Amberlite® 10 FPC22H resin (H+). As would be known to those skilled in the art, other suitable strong acid cation-exchange resins for use with embodiments of this invention are commercially available.

The anion-exchange resin can be any weak base anion-exchanger where the functional group is, for example, a tertiary amine. Suitable weak base anion exchange resins are known in the art and include, but are not limited to, resins such as Amberlite-FPA53 (OH—), Amberlite IRA-67, Amberlite IRA-95, Dowex 67, Dowex 77 and Diaion WA 30 may be used. In a particular embodiment, the strong acid cation-exchange resin is Amberlite-FPA53 (OH—) resin. As would be known to those skilled in the art, other suitable weak base anion-exchange resins for use with embodiments of this invention are commercially available.

A key to the advantages of the method described herein is the passage of the Siraitia grosvenori primary extract through a multi-column system including a plurality of columns, in series, packed with a porous adsorbent resin and the elution of fractions with Mogroside V content.

In this way, a solution of the primary extract may be passed through one or more consecutively connected columns, connected serially, and packed with polar macroporous polymeric adsorbent to provide at least one column having adsorbed mogrosides. In some embodiments, the number of columns can be two, in others, the number of columns may be three or four. While that number of columns is preferred, the number of columns may be, for example, 5 columns, 6 columns, 7 columns, 8 columns, 9 columns, 10 columns, 11 column, 12 columns, 13 columns, 14 columns or 15 columns. As used herein, the term “columns” is used interchangeably with the term “fractions”.

In certain embodiments, the first column in the sequence can be a “catcher column”, which is used to adsorb certain impurities that have higher adsorption rates and faster desorption rates than most mogrosides. In some embodiments, the “catcher column” size can be about one-third the size of the remaining columns. The ratio of internal diameter to column height or so-called “diameter: height ratio” of the columns may be between about 1:1 to about 1:100, such as, for example, about 1:2, about 1:6, about 1:10, about 1:13, about 1:16, or about 1:20. In a particular embodiment, the diameter: height ratio of the column is about 1:3. In yet another embodiment, the diameter: height ratio is about 1:8. In still another embodiment, the diameter: height ratio is about 1:15.

The polar macroporous polymeric adsorbent may be any macroporous polymeric adsorption resins capable of adsorbing mogrosides, such as, for example, the Amberlite® XAD series (Rohm and Haas), Diaion® HP series (Mitsubishi Chemical Corp), Sepabeads® SP series (Mitsubishi Chemical Corp), Cangzhou Yuanwei YWD series (Cangzhou Yuanwei Chemical Co. Ltd., China), or the equivalent. In a most preferred form, the column is a macroporous adsorption resin type: D101, with a specific surface area: 480-520 m²/g and average pore size: 13-14 nm. The individual columns may be packed with the same resin or with different resins. The columns may be packed with sorbent up to from about 75% to about 100% of their total volume.

The solvent that carries the mogroside solution through the column system may comprise water, alcohol or a combination thereof (for example, an aqueous alcoholic solvent). The water to alcohol ratio (vol/vol) in the aqueous alcoholic solvent may be in the range of about 99.9:0.1 to about 60:40, such as, for example, about 99:1 to about 90:10. The specific velocity (SV) can be from about 0.3-1 to about 1.5-1, such as, for example, about 1.0 hour-1. Preferably, alcohol is selected from the group consisting of methanol, ethanol, n-propanol, 2-propanol, 1-butanol, 2-butanol and mixtures thereof.

The alcohol can be selected from, for example, methanol, ethanol, n-propanol, 2-propanol, 1-butanol, 2-butanol and mixtures thereof.

Mogrosides contained with the solution of the primary extract become adsorbed with the pores of the selected resin, packed inside the columns upon passage of the solution through the plurality of columns, in series. Desorption, i.e. release of the trapped mogrosides, can be carried out with an aqueous alcohol solution. Suitable alcohols include methanol, ethanol, n-propanol, 2-propanol, 1-butanol, 2-butanol and mixtures thereof. In a particular embodiment, the aqueous alcoholic solution can contain between about 30% to about 70% alcohol content, such as, for example, between about 40% to about 70%, about 50% to about 65%, about 58%, about 59%, about 60%, about 65%, about 70%. In a particular embodiment, the aqueous alcoholic solution contains between about 55% to about 75% ethanol. A SV between about 0.5 hour-1 to about 3.0 hour-1, such as, for example, between about 1.0 hour-1 and about 1.5 hour-1 can be used.

During desorption/elution from the end of the series connected columns, samples are periodically taken (for example 100 ml to 500 ml at a time) and are tested/analyzed for sweetness to determine when the columns are “clean” of the desired mogrosides.

A key aspect of the viability of this method for the extraction and purification of mogrosides (in particular Mogroside V) is that the resin columns can be regenerated and reused. Previously, such a plurality of macroporous columns in series had never been used for mogroside extractions. So, upon complete passage through the one or more columns, the resins can optionally be washed with a washing solution to remove impurities. Suitable washing solutions include an aqueous or alcoholic solution, where the aqueous solution can contain any suitable acid or base to arrive at the desired pH.

The water to alcohol ratio (vol/vol) in the aqueous alcoholic solution is in the range of about 99.9:0.1 to about 60:40. Multiple washes of the columns with the same, or different, wash solutions can be performed, followed by wash(es) with water until the pH of the effluent from the one or more columns is about neutral (i.e., has a pH from about 6.0 to about 7.0). In a particular embodiment, the resins of the one or more columns is washed sequentially with one volume of water, two volumes of NaOH, one volume of water, two volumes of HCl, and finally with two volumes of water until it reached a neutral pH. The elution of impurities is carried out from two or more consecutively connected columns, as they are provided serially.

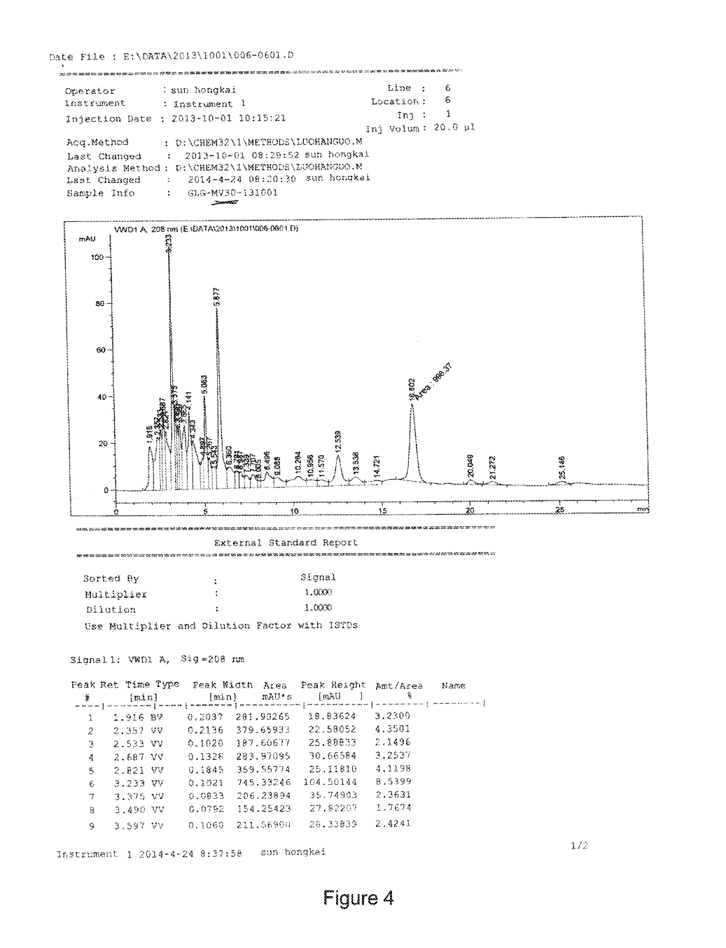

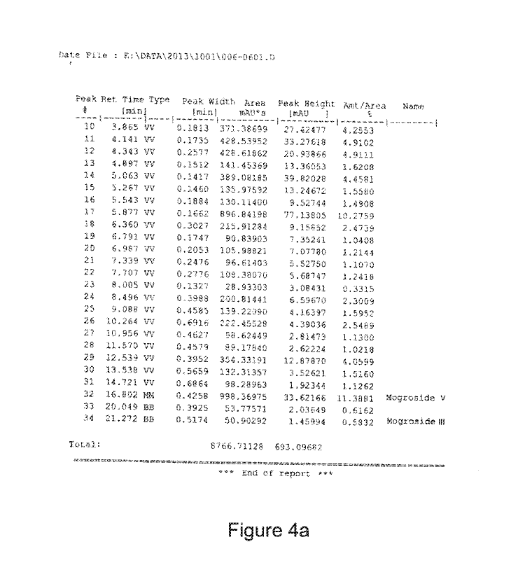

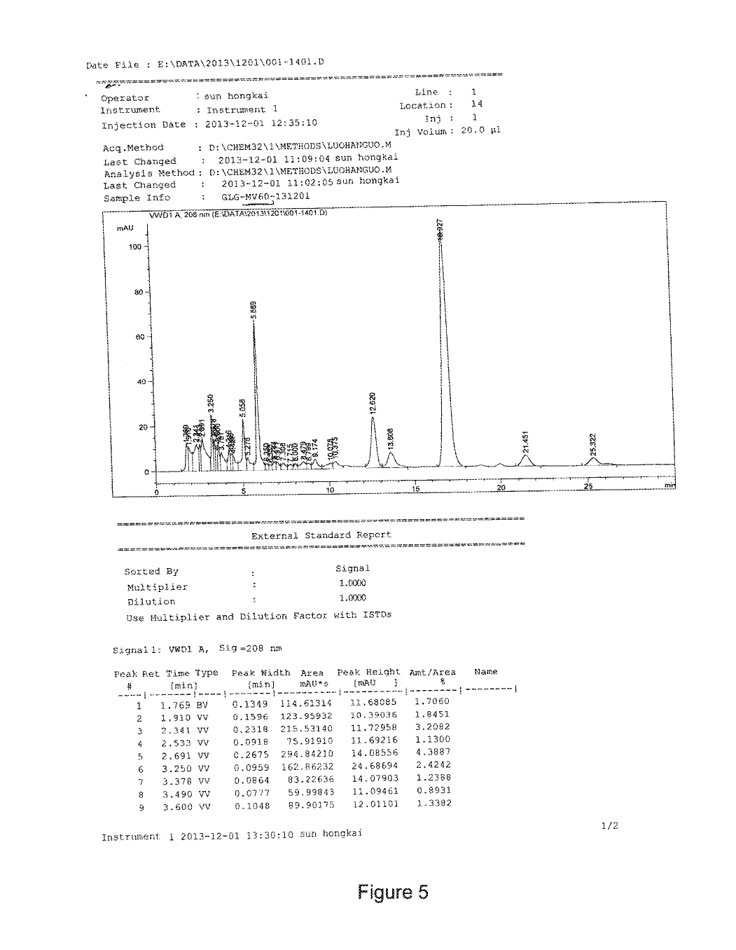

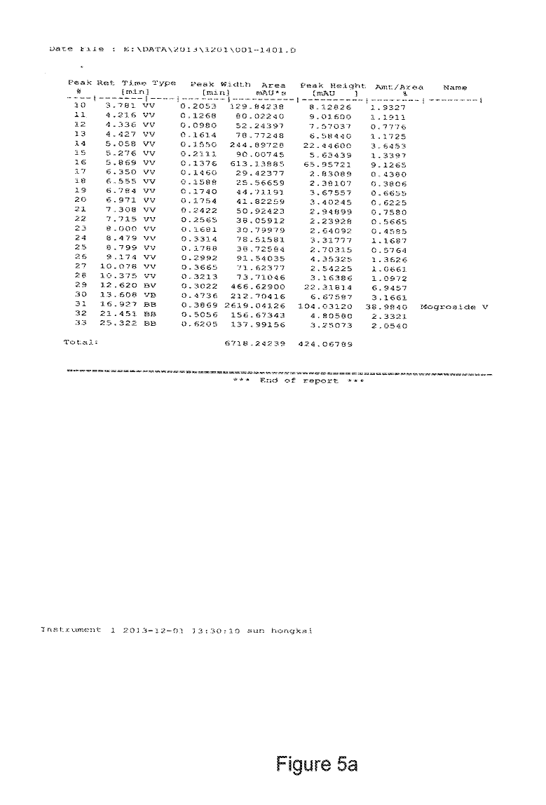

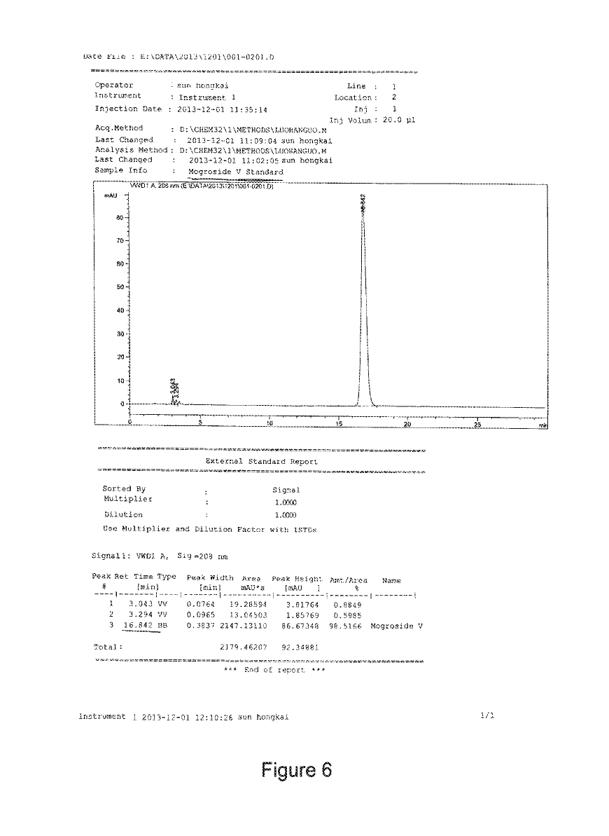

The Mogroside V elution can determined experimentally by HPLC or HPLC/MS. For example, chromatographic analysis can be performed on a HPLC/MS system comprising an Agilent 1200 series (USA) liquid chromatograph equipped with binary pump, autosampler, thermostatted column compartment, UV detector (210 nm), and Agilent 6110 quadrupole MS detector interfaced with Chemstation data acquisition software. The column can be a “Phenomenex Prodigy 5u ODS3 250×4.6 mm; 5 μm (P/No. 00G-4097-E0)” column maintained at 40° C. The mobile phase can be 30:70 (vol/vol.) acetonitrile and water (containing 0.1% formic acid) and the flow rate through the column can be 0.5 mL/min. The mogrosides (specifically also Mogroside V) can be identified by their retention times in such a method, which are generally around 16.8 minutes for Mogroside V. One of skill in the art will appreciate that the retention times for the various mogrosides given above can vary with changes in solvent and/or equipment. FIG. 4 shows the HPLC elution profile of, for example, sample GLG-MV30-131001. This extract comprises 30 wt % of Mogroside V, post extraction. Mogroside V is shown to elute at 16.802 minutes with an area of 998.37 mAU. and peak height of 33.621 mAU. FIG. 5 shows the HPLC elution profile of, for example, sample GLG-MV60-131201. This extract comprises 60 wt % of Mogroside V, post extraction. Mogroside V is shown to elute at 16.927 minutes with an area of 2619.04 mAU. and peak height of 104.031 mAU. FIG. 6 shows the HPLC elution profile of, a Mogroside V standard: shown to elute at 16.842 minutes with an area of 2147.13 mAU. and peak height of 86.673 mAU.

Those of skill in the art will also recognize that one or more of the “decolorizing”, “second adsorption” and “deionization” steps, described herein may be omitted. Those experienced in art will also understand that although the process described herein assumes certain order of the described steps, this order can be altered in some cases.

The following represents more preferred comments on reaction conditions:

### Cleaning Procedure

Luo Han Guo fruits are preferably cleaned in a moving water bath equipped with air agitator. The water bath may have two compartments: the first compartment is for initial wash, whereas the second compartment is for further washing. The fruits are moved through the two compartments by the force of moving water propelled by air agitation from below. The fruits are thoroughly washed to remove all adhering dirt. The washing water should not contain disinfectant solutions, detergents or chlorine.

### Crushing/Cracking Open the Luo Han Guo Fruits

The outer peel (shell) of the Luo Han Guo fruits is gently broken by a mashing apparatus, or any other processing technology generally available in the art. Luo Han Guo fruits contain a large amount of seeds, and it is important to not smash the seeds because it would cause a bitter flavor in the final product. The extraction is based on peel and pulp, not including any seed.

### Saccharification of Luo Han Guo Fruits

The function of this saccharification step is to use enzymes (for example, polygalacturonase) to shorten the fresh Luo Han Guo fruits ripening time, decompose the pectin and polysaccharides and increase the Mogroside V content. By way of example, crushed fresh Monk fruit is mixed into a saccharifying solution and stirred well with a stirrer, placed at holding temperature, the described saccharifying solution is obtained by polygalacturonase dissolved in the ultrapure water treated through reverse osmosis wherein the polygalacturonase accounts for 5˜10 wt % c in the weight of fresh Monk fruit, and wherein ultrapure water volume is about 2000 L, and wherein holding temperature is about 25˜35° C., and wherein standing time is about 4-5 hours.

### Water Extraction and Concentration

In this step, first the saccharified fresh fruits are extracted with ultrapure water; then they are extracted and filtered (for example, with water reflux). It is preferred the filtering step is done four times, and then the extraction filtrate obtained (from the four times) are merged; and then concentrated at reduced vacuum.

Most preferably, the described extraction and filtration with water reflux (four times) are conducted at a temperature over 85˜100° C. above; for the first time, four times water of the feed volume is added, the extraction lasts about 2 hours, and then filtration is conducted; filter residues enter the second extraction, to which two times water of the feed volume is added, the extraction lasts about 1.5 hours, and then filtration is conducted; filter residues enter the third extraction, to which three times water of the feed volume is added, extraction time lasts about 1 hour, and then filtration is conducted; filter residues enter the fourth extraction, to which value 1 time of the feed volume is added, extraction lasts about 0.5 hour, and then filtration is conducted; the said concentration under reduced vacuum means concentration at below 65° C. to 5 times of the feed weight of fresh Monk fruit. Tap water in many regions can be used in this step.

### Sedimentation and Centrifugation

The concentrated extraction filtrate is the centrifuged at high speed and sedimentated to produce centrifugate, wherein the centrifuge speed is preferably from about 3000˜5000 r/min, and wherein sedimentation and centrifugation time is preferably 1-1.5 hours.

### Adsorption and Separation

In this next steep, a plurality of macroporous resin chromatographic columns, in series connection, are used for separation. More preferably, a macroporous adsorption resin is mounted into the resin column, with resin columns in series connection constituting a “resin column set”, and the feed liquor passes through the columns for adsorption. After adsorption, resolution is made with alcohol (for example 60-75% of ethanol) and the resolving solution is collected. In this resolving process, resolutions are conducted by stages based on the volume of solution collected and an analysis is made to respectively for each fraction. The next processing procedure corresponding to the resolving solution will be based on the content of Mogroside V in the solution at each stage. As such, the eluted fraction is analyzed for the solid content, the mogrosides content and Mogroside V content. If the Mogroside V content is above a desired amount (such as, for example, above 50 or 55%), the next step process will be continued. It has found that four columns connected in series is the most efficient way to achieve a desired 60 wt % of Mogroside V.

At this step and critically, multiple sets of said resin columns are connected in series, each set acting to absorb in order from the first column to the fourth column until the eluant of the fourth column releases an extract/solution with the desired sweetness. Adsorption between the sets is subject to selective adsorption and chromatographic separation based on product requirements.

It is most preferred that the feed liquor to upper column has a flow range of 0.1˜5B V/h, after the end of adsorption, the water washing flow range is 0.1˜5 BV/h until the effluent water becomes colorless, such period lasts 5˜20 h.

What preferable is, after water washing, 70% alcohol is used for resolution, and the resolving solution with alcohol content ≧50% is collected; the described resolution by stages means: when the resolving solution carrying particles of different size passes through the resin, the particles of different size pass through the resin along with the leakage solution at different periods, the leaking solution at different periods is resolved respectively, and HPLC analysis is performed on the resolving solution collected at all stages.

### Purification

At this stage, colour salt and other impurities may be removed by a variety of methods including via ion exchange resins, membrane filtration and activated carbon treatment

A selected acidic cation exchange resin—001×16 and basic anion exchange resin—D301R may be mounted into the resin column respectively. The resolving solution requiring anion and cation exchange as determined in the step above are treated by the anion and cation exchange resin respectively. The purified solution from the anion and cation exchange resin columns is collected. After water washing, all effluent solutions are merged. Preferably, the acidic cation exchange resin is 001×16 benzene ethylene strongly acidic cation, basic anion exchange resin is D301R strongly basic anion, and the flow of resolving solution in processing is =2˜3 BV/h.

Using the special anion and cation resins is preferred for purification. While other resins may also work, anion and cation resins are most efficient for this process. This step remove the colors, slats and improve the sensory profile of the final product.

### Concentration and Recovery

The resolving solution resulting from above steps is then concentrated at reduced vacuum at a temperature <70° C. into concentrated solution from which alcohol (for example ethanol) is recovered, wherein the concentrated solution is concentrated to be, preferably, 0.5˜1 times of the feed weight of fresh fruit.

### Purification

Preferably, a selected silica gel (for example C18) is mounted into the resin column and the above-prepared concentrated solution is diluted by purified water and passed through the silica gel column to remove impurities and homologs of mogroside. All effluent solutions are collected. Other means are possible including membranes, chemical methods, and partitions.

Preferably, the silica gel has a specific surface of 200˜300 m2/g and a pore volume of 0.70˜0.90 ml/g, after the said dilution, the concentrated solution passes through reverse phase silica gel column for separation and elution until there is no sweetness, upon testing. HPLC or other analyses may be employed.

### Concentration and Drying

The resulting material may be dried by using a conventional spray drying unit or by using a conventional spray agglomeration unit, rotary evaporation, spray drying or other means. Or the material previously prepared may be used as-is.

In one aspect, the solution may proceed through dynamic dealcoholization and dewatering and concentration by organic membranes, the secondary concentration, reduced-pressure distillation and concentration to a nominal concentration of 10˜18 baume degrees, and finally spray drying to come to the final product, that is, the Mogroside V extract.

Compared with the existing technologies, the method of the invention has the following advantages and effects: (1) selective adsorption: the macroporous resin column acts to absorb in the form of four resin columns connected in series into a set, and multiple sets of resin column connected which allows the actual operation to be free from influence of external factors such as periodic cleaning, regeneration and raw and auxiliary materials, in this way, production efficiency can be improved, service life of resin can be extended, and product quality can be ensured stably. (2) Collection of resolving solution by stages: resolving solution is collected by stages, and merged for processing in batches depending on the content of Mogroside V tested, rather than the usual way of all resolving solution being collected together for processing. In this way, separation of the high-Mogroside V batches and medium and low-Mogroside V batches of products can be ensured so as to guarantee product quality and shorten the process cycle, as well as provide corresponding products selectively according to the needs. (3) Significant integrative effect: the purification process of this invention achieves the effect of enrichment by stages for a variety of mogrosides in bed, and the extracted Mogroside V has high purity, excellent color and pure taste.

Within the scope of the present invention, it is preferred to extract and purify Mogroside V. The Mogroside V content in the final product will most preferably reach up 60 wt % by dried weight. This content is considered an upper ideal. Achieving 70 wt % or 90 wt % is possible using the method of the invention but the cost-benefit analysis suggests that this content is commercially viable and achieves the desired sweetening. If cost is no issue in its application, the method of the invention could achieve over 90 wt % content of Mogroside V. The Mogroside V content is the most important factor for the product sensory taste profile. The focus of the method of the invention is to economically and practically extract Mogroside V. It has been found that when the Mogroside V content extracted is at about 50 to 60 wt %, the total mogrosides content in the extracted product is about 85-90 wt %. In this way, a highly desirable, commercial product is created. So, while the term “high” is used herein in regards to Mogroside V content, it is intended also to encompass an extract/composition which comprises a Mogroside V content of from about 50 to 60 wt %, in one preferred form.

### Natural Sweetener Compositions

Natural sweetener compositions of the present invention comprise the Mogroside V extract, described and claimed herein. This Mogroside V extract has a taste profile comparable to sugar and may be blended into a variety of natural sweetener compositions. Such a composition can be added, for example, to beverages and food products to satisfy consumers looking for a sweet taste. There is provided herein a process to selectively extract Mogroside V from fresh Monk Fruit in a manner which reduces negative properties in order to customize sweetening goals.

### Formulations

A further aspect of the present invention provides a solution to the problem of reduction of sugar intake while not sacrificing sweet taste. The present invention takes full advantage of the appreciated properties of Mogroside V, while creating an extract free or substantially free of bitter taste, and which can be produced without extraction/cost difficulties. The present invention not only overcomes the disadvantages of high calories and health effects due to excessive intake of white sugar, but also utilizes fully the advantage of Mogroside V in being purely natural, and having a high sweetness, and good safety and stability; and the compounded sweetener has a better mouth-feel and fresher taste, and is safer and more convenient for use, meeting people's demands for reducing calories in diets.

The Mogroside V extract and natural sweetener composition thereof of the present invention, for use as a sweetener, may additionally comprise other steviol glycosides, including, Reb A, Reb C, Reb B, STV, Reb D and Reb M. Said composition, in one aspect, may also comprise a secondary sweetening component as described further below.

Preferably compositions comprise Mogroside V, which is extracted and purified from Siraitia grosvenori according to the methods of the invention and (based on dry leaf weight) and:

from 13-20% by weight Rebaudioside A (also called RebA)

from 1-2% by weight Rebaudioside C (also called RebC)

under 5% by weight STV

Preferably such compositions, comprise Mogroside V, which is extracted and purified from Siraitia grosvenori according to the methods of the invention and (based on dry leaf weight) and:

from 14-17% by weight Rebaudioside A

from 1-3% by weight Rebaudioside C

from 2-3% by weight STV

Preferably such compositions, comprise Mogroside V, which is extracted and purified from Siraitia grosvenori according to the methods of the invention and (based on dry leaf weight)

from 13-20% by weight Rebaudioside A

from 1-2% by weight Rebaudioside C

under 5% by weight STV

from 1-2% Rebaudioside D

Preferably such compositions, comprise Mogroside V, which is extracted and purified from Siraitia grosvenori according to the methods of the invention and (based on dry leaf weight) and:

from 14-17% by weight Rebaudioside A

from 1-3% by weight Rebaudioside C

from 2-3% by weight STV

from 1-2% Rebaudioside D

Preferably such compositions, comprise Mogroside V, which is extracted and purified from Siraitia grosvenori according to the methods of the invention and (based on dry leaf weight) and

from 4-7% by weight Rebaudioside C

from 2-5% by weight Rebaudioside A

from 0.5-3% by weight STV

Preferably such compositions, comprise Mogroside V, which is extracted and purified from Siraitia grosvenori according to the methods of the invention and (based on dry leaf weight) and

from 5-6% by weight Rebaudioside C

from 3-4% by weight Rebaudioside A

from 1-2% by weight STV

Preferably such compositions, comprise Mogroside V, which is extracted and purified from Siraitia grosvenori according to the methods of the invention and (based on dry leaf weight) and

from 24-58% by weight Rebaudioside A

from 24-60% STV

Preferably such compositions, comprise Mogroside V, which is extracted and purified from Siraitia grosvenori according to the methods of the invention and a blend of Stevioside extract and RebA extract wherein the ratio of RebA extract to Stevioside extract is between about 12:1 to about 1:12 or from about 12:1 to about 2:1. An even more preferred ratio for the ratio between Reb A extract and STV extract is between about 9:1 and about 1:9. A further preferred ratio for the ratio between Reb A extract and STV extract is between about 5:1 and about 1:5. Another preferred ratio for the ratio between Reb A extract and STV extract is between about 4:1 and about 1:4. Another preferred ratio for the ratio between Reb A extract and STV extract is between about 3:1 and about 1:3. Another preferred ratio for the ratio between Reb A extract and STV extract is between about 2:1 and about 1:2. The RebA extract and the Stevioside extract may (each or both) have a purity between about 60% to about 97.5% purity.

In an alternative embodiment, the natural sweetener compositions of the present invention additionally comprise a secondary sweetening component. The secondary sweetening component is preferably selected from the group consisting of sucrose, erythritol, fructose, glucose, maltose, lactose, corn syrup (preferably high fructose), xylitol, sorbitol, or other sugar alcohols, inulin, miraculin, monetin, thaumatin and combinations thereof, and also non-natural sweeteners such as aspartame, neotame, saccharin, sucralose and combinations thereof. The natural sweetener compositions may be used alone or in combination with other secondary sweeteners, as described herein, and/or with one or more organic and amino acids, flavours and/or coloring agents.

Preferably such compositions of the invention comprise Mogroside V, which is extracted and purified from Siraitia grosvenori according to the methods of the invention blended with STV and RebA, (the latter two with a 95% purity or higher); and optionally with

2) one or more natural sweeteners, preferably crystalline fructose; and optionally with

3) one or more flavouring agents, preferably thaumatin.

Preferably such compositions of the invention comprise Mogroside V, which is extracted and purified from Siraitia grosvenori according to the methods of the invention blended with

A: One or more Stevia extracts selected from the group consisting of: RebA (20, 40, 60, 80, 97, 97, 98, 99); STV (20, 40, 60, 80, 95, 97, 98, 99); RebC (20, 40, 85, 90, 95, 97, 98, 99); RebB (95/97); RebD (95/97), and steviolbioside (95/97); and

B: None, one or more than one natural sweeteners selected from the group consisting of: High Fructose Syrup, Crystalline Fructose, Sugar, Isomaltulose, Lactulose, Soybean Oligosaccharide, fructooligosaacharide, Lactosucrose, Xylooligosaacharide, Erythritol, Xylitol, Sorbitol, Mannitol, Maltitol, Lactitol, Isomaltitol, and Glycyrrhizin; and

C: None, one or more than one flavors selected from the group consisting of: Thaumatin, Monellin, Miraculin, Glycine, Amino Acids, L-Glutamic Acid, and fragrances.

Compositions containing steviol glycosides-Mogroside V blends may be processed using known methods to modify particle size and physical form. Methods such as agglomeration, spray-drying, drum drying and other forms of physical processing may be applied to adjust particle size in order to deliver better flow, hydration, or dissolution properties. The compositions may be provided in liquid forms, optionally containing one or more preservatives and/or processing aids, for ease-of-use in specific applications. Compositions containing steviol glycosides-Mogroside V blends may be co-processed with bulking agents such as maltodextrins and similar compounds to deliver products with controlled sweetness, dosing, potency, and handling properties.

The sweetener compositions of the present invention may be used in the preparation of various food products, beverages, medicinal formulations, chemical industrial products, among others. Exemplary applications/uses for the sweetener compositions include, but are not limited to: (a) food products, including canned food, preserved fruits, pre-prepared foods, soups, (b) beverages, including coffee, cocoa, juice, carbonated drinks, sour milk beverages, yogurt beverages, meal replacement beverages, and alcoholic drinks, such as brandy, whisky, vodka and wine; (c) grain-based goods—for example, bread and pastas, cookies, pastries, whether these goods are cooked, baked or otherwise processed; (d) fat-based products—such as margarines, spreads (dairy and non-dairy), peanut butter, peanut spreads, and mayonnaise; (d) Confectioneries—such as chocolate, candies, toffee, chewing gum, desserts, non-dairy toppings (for example Cool Whip®), sorbets, dairy and non-dairy shakes, icings and other fillings, (e) drug and medicinal formulations, particularly in coatings and flavourings; (f) cosmetics and health applications, such as for sweetening toothpaste; and (g) seasonings for various food products, such as soy sauce, soy sauce powder, soy paste, soy paste powder, catsup, marinade, steak sauce, dressings, mayonnaise, vinegar, powdered vinegar, frozen-desserts, meat products, fish-meat products, potato salad, bottled and canned foods, fruit and vegetables.

The natural sweetener compositions of the present invention may be formulated into premixes and sachets. Such premixes may then be added to a wide variety of foods, beverages and nutraceuticals. The purified natural sweetener compositions may, in one preferred form, be table top sweeteners.

While the forms of processes and compositions described herein constitute preferred embodiments of this invention, it is to be understood that the invention is not limited to these precise forms. As will be apparent to those skilled in the art, the various embodiments described above can be combined to provide further embodiments. Aspects of the present composition, method and process (including specific components thereof) can be modified, if necessary, to best employ the systems, methods, nodes and components and concepts of the invention. These aspects are considered fully within the scope of the invention as claimed. For example, the various methods described above may omit some acts, include other acts, and/or execute acts in a different order than set out in the illustrated embodiments.

Further, in the methods taught herein, the various acts may be performed in a different order than that illustrated and described. These and other changes can be made to the present systems, methods and articles in light of the above description. In general, in the following claims, the terms used should not be construed to limit the invention to the specific embodiments disclosed in the specification and the claims, but should be construed to include all possible embodiments along with the full scope of equivalents to which such claims are entitled. Accordingly, the invention is not limited by the disclosure, but instead its scope is to be determined entirely by the following claims.

All publications, patents and patent applications mentioned in the specification are indicative of the level of those skilled in the art to which this invention pertains. All such publications, patents and patent applications are incorporated by reference herein for the purpose cited to the same extent as if each was specifically and individually indicated to be incorporated by reference herein.

The following example illustrates a preferred but non-limiting embodiment of the present invention.

### EXAMPLES

### Example 1

Soak in two batches, one batch of 2000 g Monk fruits, and another batch of 1800 g Monk fruit. Two batches are absorbed and resolved by going through the macroporous adsorption resin columns respectively, and then the resolving solution are merged as a batch and classified and combined based on the detected Mogroside V content for purification separately, thereby three small batches of finished products are produced.

1.1 Take and Put 2000 g Monk Fruits into 20000 ml Water at a Temperature of 80° C., Soaking for 5 Hours to Get Soak Solution of 20500 ml Through a Filter.

Macroporous resin adsorption and resolution: the resolving solution is removed by level (resolving solution is summarized in Table 1 below)

| | | | Total | | | |
|---|---|---|---|---|---|---|
| | | Solid | Mogrosides | Mogroside | Mogroside | Removal |
| | | content | content | V content | V content | amount |
| No. | Batch No. | (%) | (g) | (%) | (g) | (ml) |
| 1 | DD-30-20131121 | 0.35 | 2.5165 | 37.05 | 0.932 | 719 |
| 2 | DD-55- | 0.95 | 0.931 | 62.03 | 0.5778 | 98 |
| | 20131121-1 | | | | | |
| 3 | DD-55- | 3.2 | 2.8757 | 58.96 | 1.6955 | 90 |
| | 20131121-2 | | | | | |
| 4 | DD-55- | 1.2 | 1.461 | 31.52 | 0.4463 | 118 |
| | 20131121-3 | | | | | |
| 5 | DD-55- | 5.32 | 6.384 | 42.68 | 2.725 | 120 |
| | 20131121-4 | | | | | |
| 6 | DD-55- | 3.57 | 4.07 | 21.94 | 0.893 | 114 |
| | 20131121-5 | | | | | |
| 7 | DD-55- | 0.09 | 0.0144 | 31.84 | 0.0458 | 160 |
| | 20131121-6 | | | | | |
| 8 | DD-45- | Not | None | 17.36 | None | 167 |
| | 20131121-1 | Detected | | | | |
| 9 | DD-5-20131121-1 | Not | None | Not | None | 550 |
| | | Detected | | Detected | | |
| 10 | Total | | 17.3216 | | 7.3154 | 2136 |
| 11 | Average content | | | 42.233 | | |
| | of Mogroside V | | | | | |

### 1.2 the Second Batch Process is Controlled as Follows:

With reference to FIG. 1:

a. Take and put 1800 g Monk fruits into 18000 ml water at a temperature of 80□, soaking for 5 hours to get soak solution of 18500 ml through a filter.

b. Macroporous resin adsorption and resolution: the resolving solution is removed by level (resolving solution is summarized in Table 2 below)

TABLE 2

| | | | Total | | | |
|---|---|---|---|---|---|---|
| | | Solid | Mogrosides | Mogroside | Mogroside | Removal |
| | | content | content | V content | V content | amount |
| No. | Batch No. | (%) | (g) | (%) | (g) | (ml) |
| 1 | DD-50- | Not | None | Not | None | 93 |
| | 20131125-1 | Detected | | Detected | | |
| 2 | DD-50- | 2.62 | 2.2794 | 41.81 | 0.9530 | 87 |
| | 20131125-2 | | | | | |
| 3 | DD-50- | 7.2 | 6.552 | 61.80 | 4.049 | 91 |
| | 20131125-3 | | | | | |
| 4 | DD-50- | 5.7 | 5.3295 | 38.11 | 2.0311 | 93.5 |
| | 20131125-4 | | | | | |
| 5 | DD-50- | 2.43 | 2.2842 | 59.44 | 1.3577 | 94 |
| | 20131125-5 | | | | | |
| 6 | DD-50- | 1.18 | 1.062 | 26.66 | 0.2831 | 90 |
| | 20131125-6 | | | | | |
| 7 | DD-50- | 0.5 | 0.475 | 21.7 | 0.1030 | 95 |
| | 20131125-7 | | | | | |
| 8 | DD-50- | 0.18 | 0.1584 | 20.92 | 0.0331 | 88 |
| | 20131125-8 | | | | | |
| 9 | DD-50- | 0.12 | 0.1104 | 28.19 | 0.0311 | 92 |
| | 20131125-9 | | | | | |
| 10 | DD-45- | 0.03 | 0.0432 | 25.93 | 0.0112 | 144 |
| | 20131125-1 | | | | | |
| 11 | DD-45- | 0.02 | 0.0268 | 13.48 | 0.0036 | 134 |
| | 20131125-2 | | | | | |
| 12 | DD-45- | Not | None | Not | None | 175 |
| | 20131125-3 | Detected | | Detected | | |
| 13 | DD-5-20131125-1 | Not | None | Not | None | 387 |
| | | Detected | | Detected | | |
| 14 | Total | | 18.3209 | | 8.8559 | 1663.5 |
| 15 | Average content | | | 48.34 | | |
| | of mogroside V | | | | | |

### 1.3 Above Two Batches are Merged and Classified for Purification, the Test Process Control is as Follows:

a. In the above two batches of resolving solution, there are totally six batches with Mogroside V content over 40% that are merged into one batch, resolving solution totally: 580 ml, and purified solution produced: 570 ml. Batch number: TR-50/55-20131121;

b. In the above two batches of resolving solution, there are totally four batches with Mogroside V content between 30-40% that are merged into one batch, resolving solution totally: 1091 ml, and purified solution produced: 1070 ml. Batch number: TR-30/50/55-20131121;

c. In the above two batches of resolving solution, there are totally six batches with Mogroside V content between 20-30% that are merged into one batch, resolving solution totally: 623 ml, and purified solution produced: 601 ml. Batch number: TR-45/50/55-20131121;

### 1.4 Concentration

a. TR-50/55-20131121, concentrated to solid content 30%, and produced concentrated solution: 50.3 ml;

b. TR-30/50/55-20131121, concentrated to solid content 30%, and produced concentrated solution: 102.5 ml;

c. TR-45/50/55-20131121, concentrated to solid content 10%, and produced concentrated solution: 59.7 ml;

### 1.5 Carbon Treatment

a. TR-50/55-20131121, add 0.35 g active carbon to produce carbon treatment solution: 45 ml;

b. TR-30/50/55-20131121, add 0.72 g active carbon to produce carbon treatment solution: 98 ml;

c. TR-45/50/55-20131121, add 0.42 g active carbon to produce carbon treatment solution: 54 ml;

### 1.6 Drying:

a. TR-50/55-20131121 produces finished product: 9.8 g with Mogroside V content: 64.03%;

b. TR-30/50/55-20131121 produces finished product: 12.3 g with Mogroside V content: 60.63%;

c. TR-45/50/55-20131121 produces finished product: 3.1 g with Mogroside V content: 47.51%;

The total weight of above three small batches is: 25.2 g, the three small batches are mixed into one batch, the Mogroside V content calculated is: 61.02% and yield is 0.66%.

### Example 2

### Formulation Examples of MV50

| | | Times of | | | |
|---|---|---|---|---|---|
| | | Sweetness | | | Added special |
| Example | Compounding Ratio | as Sugar | Mouthfeel | Health benefits | functions |
| GLG | RA97:MV50:RC95:Sucrose = | 5.5 | pure and | low calories, | maintenance of |
| Superblend - | 1.3%:0.32%:0.6%:97.8% | | full- | avoidance of | normal content of |
| AVCS | | | bodied | obesity and | vitamins and mineral |
| | | | | prevention of the | in the body and |
| | | | | occurrence of | enhancement of |
| | | | | chronic disease | resistance |
| | | | | such as diabetes, etc. | |
| GLG | RA97:MV50:RD95:Erythrtol = | 5.2 | pure and | zero calories, | maintenance of |
| Superblend - | 1.07%:0.35%:0.6%:97.98% | | refreshing | avoidance of | normal content of |
| AVDE | | | | obesity and | vitamins and mineral |
| | | | | prevention of the | in the body and |
| | | | | occurrence of | enhancement of |
| | | | | chronic disease | resistance |
| | | | | such as diabetes, etc. | |
| GLG | RA97:MV50:RC95:RD95 = | 224 | refreshing | zero calories, | maintenance of |
| Superblend - | 60.30%:20.15%:13.48%:6.07% | | and a little | avoidance of | normal content of |
| AVCD | | | bitter | obesity and | vitamins and mineral |
| | | | | prevention of the | in the body and |
| | | | | occurrence of | enhancement of |
| | | | | chronic disease | resistance |
| | | | | such as diabetes, etc. | |
| GLG | RA97:MV50:RC95:RD95:Fructose = | 6 | pure and | low calories, | maintenance of |
| Superblend - | 1.28%:0.26%:0.36%:0.2%:97.9% | | full- | avoidance of | normal content of |
| AVCDF | | | bodied | obesity and | vitamins and mineral |
| | | | | prevention of the | in the body and |
| | | | | occurrence of | enhancement of |
| | | | | chronic disease | resistance |
| | | | | such as diabetes, etc. | |

### Example 3 Sensory Testing—Understanding the Sensory Profiles of Sweetener; Dream Sweetener Blended with RA97 and MV50, RA97 and Mogroside V

Blends of the Luo Han Guo extract prepared in accordance with the method of the invention and containing 50 wt % Mogroside V with steviol glycoside composition comprising mostly Rebaudioside A were compared in sweetness and preference panel testing. In total, five test samples were evaluated against Sucrose: Dream Sweetener with RA97 And MV50; RA97; MV25; MV50; MV55

### Protocol

Samples are prepared by GLG Life Tech Corp. During evaluations all samples were served in a sequential monadic order, randomized, in individual glass jars labeled with 3-digit codes, at ambient temperature upon panelists request.

Samples were evaluated in duplicate. Five products were tested each session—five test products and sucrose as a blind control. To minimize sensory fatigue, 10 minute breaks were imposed between samples to allow the palate to recover. Furthermore, unsalted water crackers and room temperature filtered water were used as palate cleansers in between samples.

### Preparation of Samples

Samples were prepared using the SE dilution rates supplied by GLG Life Tech Corp. All samples were prepared in glass vessels using filtered water. 200 ml glasses were used to present samples. All glasses were washed to ensure they were completely odour free. They were washed in a dishwasher using hot water only and either air dried or dried with a clean, lint free linen towel. All samples were served at room temperature to enhance flavour and for ease of preparation to be comparable to the previous study.

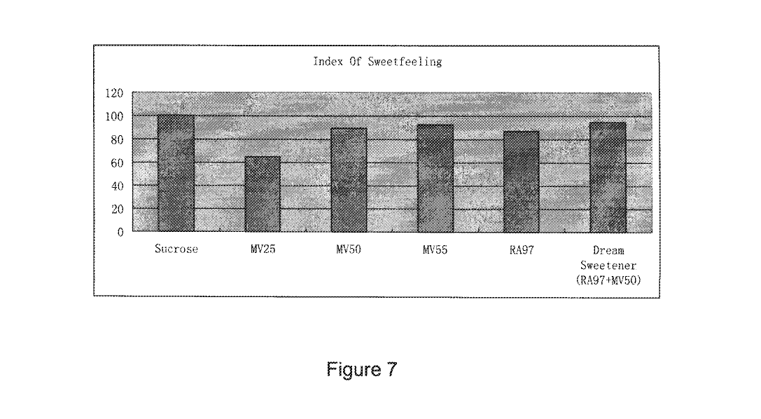

The sample comparison of their solution is shown in FIG. 7.

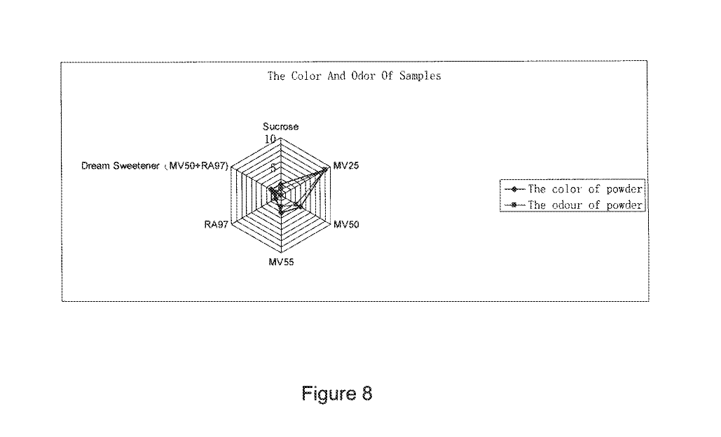

The color depth and odor of powder is shown in FIG. 8 wherein it noted that the color depth and odor are divided into 0-10 levels.

1. The color of RA97 is white, with MV55 light white, MV50 light yellow, MV25 light brown, and the color difference among samples.

2. The odor of MV25 is heavily objectionable, with MV55 and MV50 slightly smelling, and RA97 no objectionable.

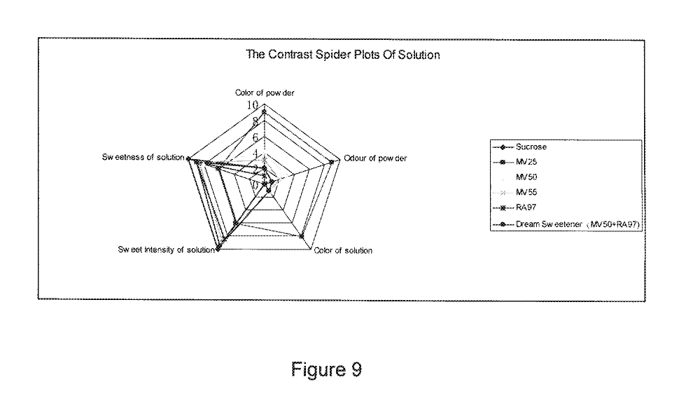

FIG. 9 shows the color shade, sweetness feeling and size of product solution wherein it noted that he color depth and odor are divided into 0-10 levels.

3. The color of MV25 solution is light brown, with the solution of MV50, MV55 and RA97 basically colorless and transparent.

4. The mouth feeling of Dream sweetener solution is better and with which has no objectionable odor. The mouth feeling of MV55 and MV50 solution is good. The mouth feeling of RA97 solution is astringent. The mouth feeling of MV25 solution is obviously mogroside odor.

5. The order of the sweetness of the test solution is dream sweetener with MV50 and RA97, RA97, MV55, MV50, MV25. And all of the solution sweetness is between 8-9 degrees without obvious difference.

6. By composite indexes comparing, the character of the dream sweetener with MV50 and RA97 is close to sucrose, while MV25 having color depth and Luo Han Guo special taste heavy. MV55 and MV50 taste better with Luo Hanguo special taste slight. RA97 tastes slight bitter and astringent.

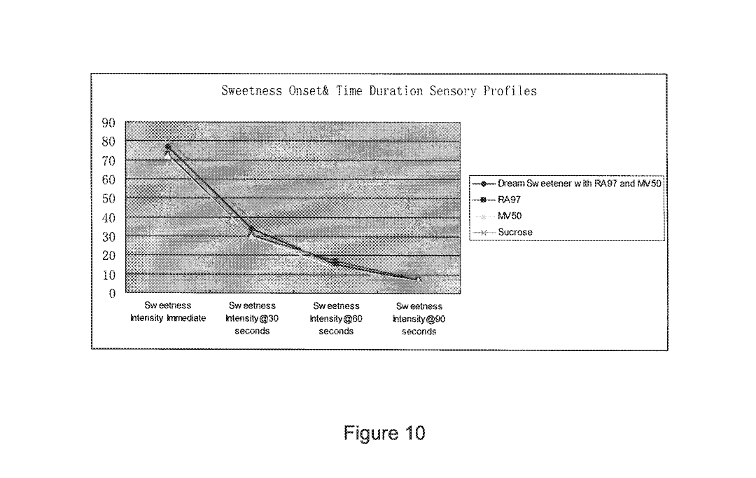

FIG. 10 shows the sweetness curve chart and time duration sensory profiles of RA97, MV50, Sucrose and dream sweetener with MV50 and RA97.

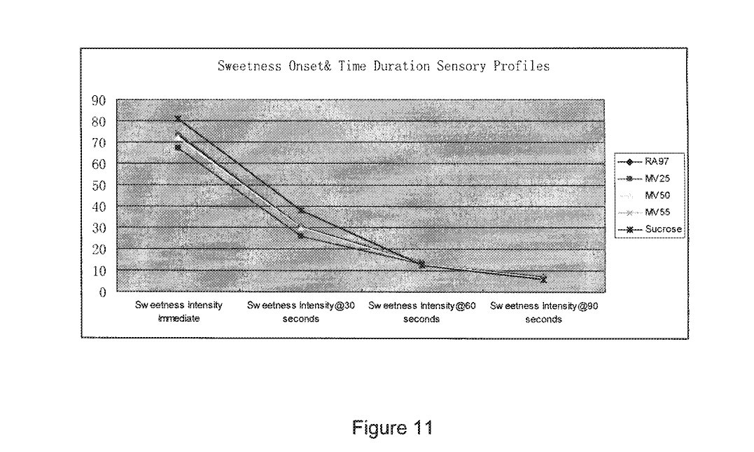

FIG. 11 shows the sweetness curve chart and time duration sensory profiles of RA97, MV25, MV50, MV55 and Sucrose.

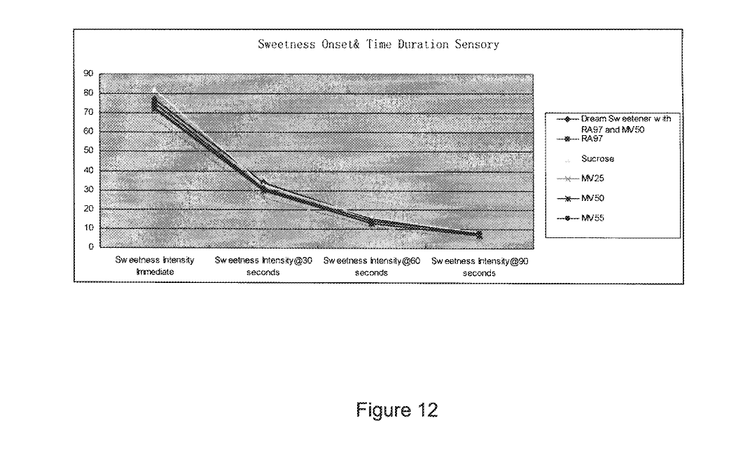

FIG. 12 shows the sweetness curve chart and time duration sensory profiles of six samples wherein it is noted that:

The comprehensive comparison chart shows that the sweetness of Dream sweetener with RA97 and MV50 is close to sucrose. The variation tendency of RA97, MV50 and MV55 is approximate. The variation tendency of MV25 is different from other products. The line sensory profiles of six samples as below.

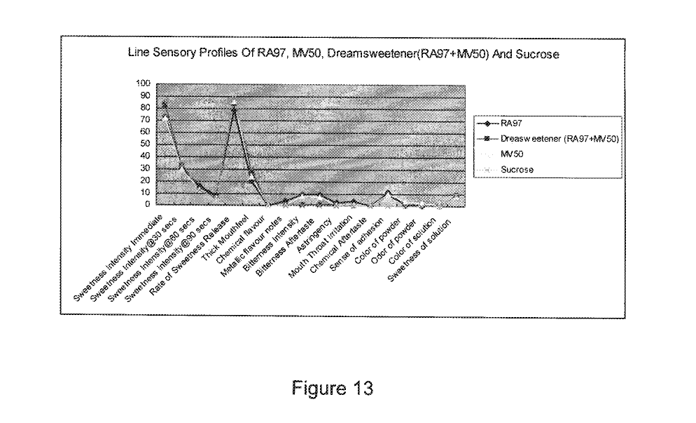

FIG. 13 shows the line sensory profiles of RA97, Dream sweetener (RA97+MV50) and Sucrose.

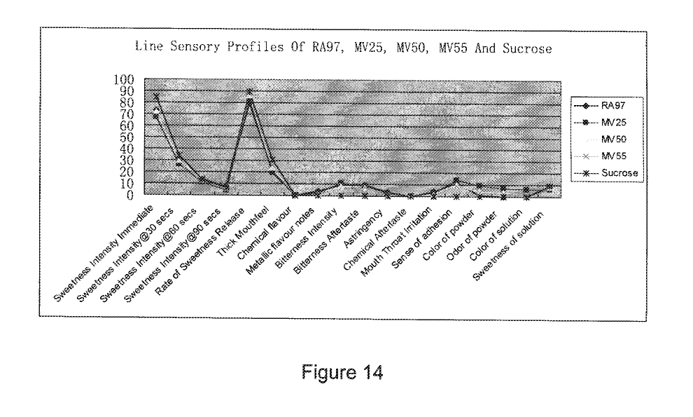

FIG. 14 shows line sensory profiles of RA97, MV25, MV50, MV55 and Sucrose.

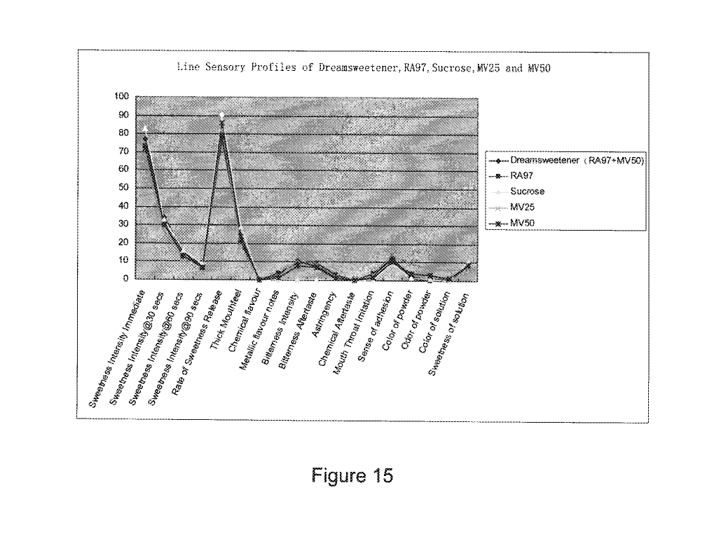

FIG. 15 shows line sensory profiles of RA97, Sucrose, Dream sweetener, MV25 and MV50 wherein it is noted that:

The comprehensive comparison chart shows that the sweetness change trend of Dream sweetener with RA97 and MV50 is close to sucrose, which of MV25 is different from other sweeteners. The indicators of RA97, MV50 and MV55 are approximate, which is better than MV25 on the thick feeling, metallic taste, tart, the smell of powder and the color of powder. The spider plots on sweetener attributes of six samples as below.

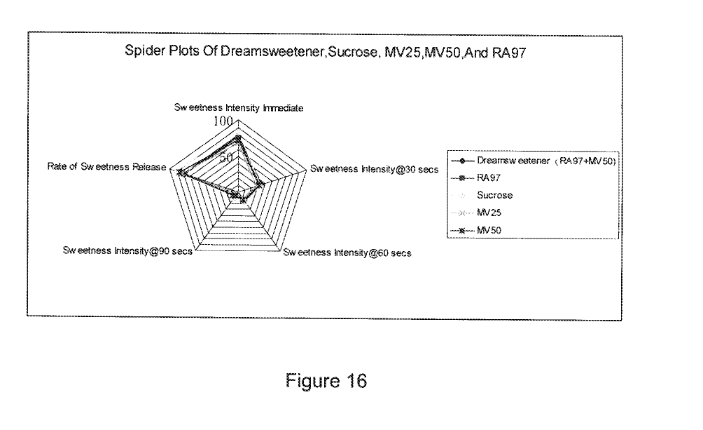

FIG. 16 shows the spider plot of Dream sweetener, RA97, Sucrose, MV25 and MV50.

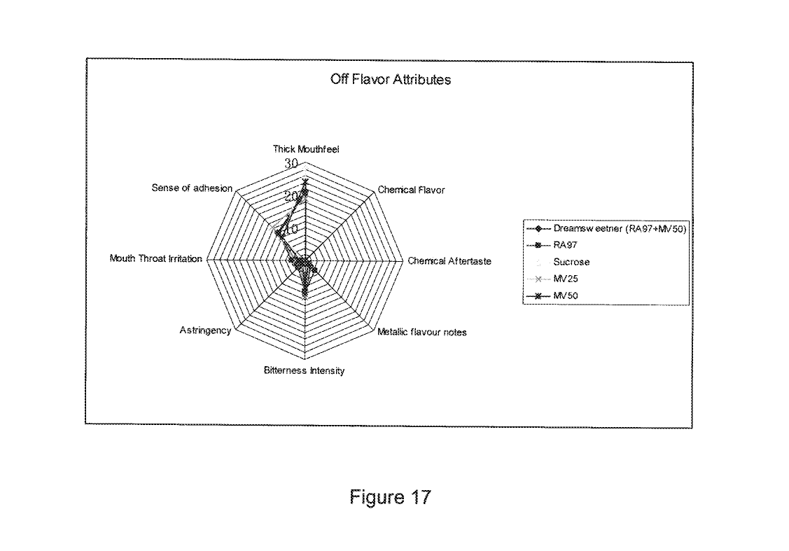

FIG. 17 shows the off flavor attributes of Dream sweetener, RA97, Sucrose, MV25 and MV50.

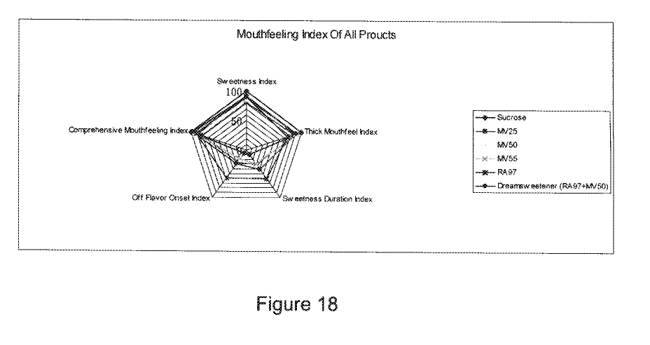

FIG. 18 shows the mouth feeling index for six samples wherein it is noted that:

1. The sweetness indicator of Dream sweetener is the most close to sucrose, of which is the most different from MV25.

2. The thick mouth feel index of the Dream sweetener is the most close to sucrose. The thick mouth feel index of RA97 is worse than the Dream sweetener.

3. The sweetness duration index of MV55 is the most close to sucrose. The duration of RA97 is the longest than other products.

4. The off flavor index of MV55 and MV50 is the most close to sucrose. The off flavor index of RA97 is higher than other products.

5. The comprehensive mouth feeling index of Dream sweetener is the most close to sucrose, which of MV55 is worse than Dream sweetener.

From this data, it can be seen that the mouth feeling of MV50 and MV55 is the best of all, and much better than MV25. The powder color of MV50 and MV55 is white and approximate to RA97. The sweetness taste of Dream sweetener with RA97 and MV50 is better than RA97 and MV50. The difference of MV55 and MV50 in color is bigger, and the difference of MV55 and MV50 in sweetness is not obvious. The powder color of MV25 is light brown, the peculiar smell of which is heavier than other products.

The following examples describe the application of Blends of Steviol glycoside/Mogroside V prepared in accordance with the method of the invention, in Food And Beverages.

### Example 4: The Application of Stevia and Mogroside V, Prepared in Accordance with the Method of the Invention, in Soft Sweets

| Raw Material | Dosage (g) |
|---|---|
| Fructose | 76 |
| Anhydrous dextrose | 210 |
| Fructooligosaccharide | 90 |
| RA97 | 2.125 |
| Mogroside V50 | 0.525 |
| Gelatin | 70 |
| Citric Acid | 3.5 |
| Natural Pigment | 0.57 |
| Natural Flavor | 4 |
| Water | 389.8 |

### Example 5: The Application of Stevia and Mogroside V in Vitamin Water

| Raw Material | Dosage (%) |
|---|---|
| RA97 | 0.007 |
| Mogroside V 50 | 0.024 |
| Fructose | 0.5 |
| Citric Acid | 0.13 |
| Vitamin C | 0.03 |
| Sodium Citrate | 0.03 |
| Salt | 0.015 |
| Xanthan gum | 0.01 |
| Natural Pigment | 0.0008 |
| Natural Flavor | 0.12 |
| Taurine | 0.05 |
| Inose | 0.008 |
| Nicotinamide | 0.001 |
| Calcium Pantothenate | 0.00014 |
| Vitamine VB6 Vitamine | 0.0001 |
| VB12 | 0.00008 |
| Potassium chlorid Zinc | 0.001 |
| gluconate | 0.005 |
| Water soluble Vitamin E | 0.004 |
| Water | Add to 100 |

### Example 6: The Application of Stevia and Mogroside V in Jelly

| Raw Material | Dosage (%) |
|---|---|
| RA97 | 0.02 |
| Mogroside V 50 | 0.015 |
| Fructose | 2 |
| Anhydrous dextrose | 13 |
| Carrageenan | 0.2 |
| Locust been gum | 0.18 |
| Konjac glucomannan | 0.2 |
| Sodium citrate | 0.18 |
| Calcium Lactate | 0.15 |
| Citric acid | 0.26 |
| Potassium Chloride | 0.1 |
| Coloring matter | Appropriate |
| Flavors and fragrances | Appropriate |
| Water | Add to 100 |

### Example 7: The application of Stevia and Mogroside V in juice milk

| Raw Material | Dosage (%) |
|---|---|
| RA97 | 0.024 |
| Mogroside V50 | 0.0075 |
| Whole milk powder | 3 |
| Skim milk powder | 1 |
| Fructose | 1.2 |
| Fruit juice, yogurt stabilizer | 0.48 |
| Modified starch | 0.1 |
| Sodium citrate | 0.03 |
| Citric acid | 0.24 |
| Malic acid | 0.12 |
| Apple juice | 0.7 |
| Apple essence | 0.04 |
| Milk flavor | 0.05 |
| Water | Add to 100 |

### Example 8: The Application of Stevia and Mogroside V in Coffee Beverage

| Raw Material | Dosage (%) |
|---|---|
| RA97 | 0.017 |
| Mogroside V 50 | 0.0042 |
| Fructose | 2.4 |
| Whole milk powder | 1 |
| Skim milk powder | 1.5 |
| Instant coffee powder | 1.5 |
| Coffee bean | 0.6 |
| Sucrose ester | 1.1 |
| Sodium bicarbonate | 0.006 |
| Flavor | Appropriate |
| Water | Add to 100 |

### Example 9: The Application of Stevia and Mogroside V in Walnut Dew

| Raw Material | Dosage (%) |
|---|---|
| RA97 | 0.015 |
| Mogroside V 50 | 0.0036 |
| Peanut | 0.5 |
| Walnut | 3.5 |
| Sucrose ester | 0.04 |
| Xanthan gum | 0.015 |
| Ethyl maltol | 0.005 |
| Stabilizer | 0.2 |
| Whole milk powder | 0.3 |
| Cyclodextrine | 0.1 |
| Fructose | 2 |
| Salt | 0.02 |
| Flavor | 0.03 |
| Water | Add to 100 |

### Example 10: The Application of Stevia and Mogroside V in Cola

| Raw Material | Dosage (%) |
|---|---|
| RA97 | 0.031 |
| Mogroside V 50 | 0.009 |
| 85% Phosphoric acid | 0.06 |
| Cola base | 0.1 |
| Fructose | 0.7 |
| Cola flavor | 0.001 |
| Bubble caramel | 0.04 |
| Flavor | 0.058 |
| Water | Add to 100 |

1. A method for purifying Mogroside V comprises passing a solution of a pre-prepared Siraitia grosvenori primary extract through a multi-column system including a plurality of columns, in series, packed with a porous adsorbent resin to provide at least one column having adsorbed mogrosides and eluting fractions with Mogroside V content from the at least one column having absorbed mogrosides to provide an eluted solution with Mogroside V content (an intermediate Mogroside V extract).

2. The method of claim 1, wherein the plurality of columns is at least two columns.

3. The method of claim 1, wherein the plurality of columns is at least three columns

4. The method of claim 1, wherein the plurality of columns is four columns.

5. The method of claim 1, wherein pre-prepared Siraitia grosvenori primary extract is prepared by i) providing fruit of a Siraitia grosvenori plant, ii) washing and mashing the fruit (while removing seeds) to produce mashed fruit, iii) saccharifying the mashed fruit to hydrolyze polysaccharides, extracting saccharified matter, filtering and concentrating the extract, and iv) centrifuging the extract, such extract being preparable for passage, as a feed liquor, through the plurality of columns, in series, packed with the porous adsorbent resin.

6. The method of claim 1, which comprises further processing of the intermediate Mogroside V extract to remove one or more of colour, salt and impurities by a means selected from the group comprising membrane filtration, ion exchange chromatography and activated carbon treatment, or any combinations thereof.

7. The method of claim 6, additionally comprising evaporating and concentrating by the intermediate Mogroside V extract via passage through at least one nanofilter or ultrafilter, followed by drying.

8-12. (canceled)

8. A method for purifying Mogroside V comprises:
passing a primary solution comprising mogrosides through a multi-column system including a plurality of columns packed with an adsorbent resin to provide at least one column having adsorbed mogrosides; and
eluting fractions with Mogroside V content from the at least one column having adsorbed mogrosides to provide an eluted solution with Mogroside V content.

9. The method of claim 8, further comprising:
removing impurities from the multicolumn system prior to eluting fractions with Mogroside V content from the at least one column having adsorbed mogrosides.

10. The method of claim 8, further comprising:
deionizing the eluted solution.

11. The method of claim 8, further comprising:
decolorizing the eluted solution with Mogroside V content to provide a first adsorption solution.

12. The method of claim 11, further comprising:
removing an alcoholic solvent from the first adsorption solution; and
passing a remaining solution without the alcoholic solvent through a column with a macroporous adsorbent to provide a second adsorption solution.

13. A product comprising Mogroside V,
wherein Mogroside V for the product is purified via passing a solution of pre-prepared Siraitia grosvenori primary extract through a plurality of columns arranged in series, the columns packed with a porous adsorbent resin, to provide at least one column having adsorbed mogrosides; and
eluting fractions with Mogroside V content from the at least one column having adsorbed mogrosides to provide an eluted solution with Mogroside V content (an intermediate Mogroside V extract).

14. The product of claim 13, wherein the product comprises one or more of at least a natural extract, a natural sweetener, a food, a beverage, and a nutraceutical.

15. The product of claim 13, wherein the plurality of columns is at least two columns.

16. The product of claim 13, wherein the plurality of columns is at least three columns.

17. The product of claim 13, wherein the plurality of columns is four columns.

18. The product of claim 13, wherein pre-prepared Siraitia grosvenori primary extract is prepared by i) providing fruit of a Siraitia grosvenori plant, ii) washing and mashing the fruit (while removing seeds) to produce mashed fruit, iii) saccharifying the mashed fruit to hydrolyze polysaccharides, extracting saccharified matter, filtering and concentrating the extract, and iv) centrifuging the extract, such extract being preparable for passage, as a feed liquor, through the plurality of columns, in series, packed with the porous adsorbent resin.

19. The product of claim 13, which comprises further processing of the intermediate Mogroside V extract to remove one or more of colour, salt and impurities by a means selected from the group comprising membrane filtration, ion exchange chromatography and activated carbon treatment, or any combinations thereof.

20. The product of claim 19, additionally comprising evaporating and concentrating by the intermediate Mogroside V extract via passage through at least one nanofilter or ultrafilter, followed by drying.
